(19) United States
(12) Patent Application Publication
(10) Pub. No.: US 2019/0216684 A1
(43) Pub. Date: Jul. 18, 2019

(54) HIPPA Compliant Monitoring System For Drug Distribution And Decreasing Drug Diversion

(71) Applicant: Bruce H. Levin, Oceanside, NY (US)

(72) Inventor: Bruce H. Levin, Oceanside, NY (US)

(21) Appl. No.: 16/247,514

(22) Filed: Jan. 14, 2019

Related U.S. Application Data
(60) Provisional application No. 62/617,001, filed on Jan. 12, 2018.

(51) Int. Cl. A61J 3/00; G06Q 50/28; A61B 5/00; G16H 20/10

(52) U.S. Cl. CPC A61J 3/007; G06Q 50/28; G06Q 10/087; G16H 20/10; A61B 5/4833

(57) ABSTRACT

The present invention provides a HIPPA compliant monitoring system for drug distribution and decreasing drug diversion. Incorporating electromagnetic tag technology, such as RF ID and other applicable technology, a system is provided to track movements of a dispensed drug, from the manufacturer to the end use. A central unit processes information received from a number of different tracking units, stores such information and processes the information with pre-determined conditions including but not limited to drug abuse, diversion, and other compliance related events. Once an abnormal event is detected by the central unit, an alert may be sent to the authorities or relevant personnel. The system also includes a one or multilevel authentication code system, to further ensure the protection of a patient's privacy during the tracking process, and to make the system HIPPA compliant.

### RELATED APPLICATIONS

This application claims priority to U.S. Provisional Patent Application Ser. No. 62/617,001 filed Jan. 12, 2018, titled the same and incorporated herein as if set out in full.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

The present invention relates generally to electromagnetic tag technology and, specifically, to the use of Radio Frequency Identification (RFID) Devices to track proper drug distribution and regimen compliance.

In typical settings, once in the possession of a patient, it is difficult to track usage and/or movement of the drugs. Such drugs may include prescription drugs that are subject to restricted and/or controlled distribution. For example, opioids and other narcotics require strict monitoring for abuse. Tracking usage of a dispensed drug not only ensures that the patient is in possession of the drug, but provides the added benefit of monitoring patient compliance with a prescribed dosing regimen.

Tracking movement of a dispensed drug also enables law enforcement to ascertain legal or illegal possession. Tracking also assists law enforcement in determining the source of the drug.

However, traditional supply chain tracking approaches are insufficient for tracking goods outside the supply chain, such as dispensed drugs. What is needed is a system for tracking restricted-distribution goods that deters diversion by providing for tracking of goods after dispensing to a consumer.

### BRIEF SUMMARY OF THE INVENTION

In some embodiments, systems and methods of the invention are operable for prescription drug tracking as described herein. Drug tracking includes receiving indicia associated with a dispensed prescription and retrieving prescription data associated with the indicia. Tracking also includes retrieving prescription data and determining whether the dispensed prescription was diverted. An indication of whether the dispensed prescription was diverted may also be communicated to a prescriber, pharmacy, manufacturer and/or law enforcement.

In one embodiment, the present invention relates to methods and systems of patient compliance and monitoring as described herein. In other embodiments, patient compliance and monitoring include receiving patient information, and prescription data associated with the patient information.

A prescriber, pharmacy, manufacturer, treating center, law enforcement, and other verifiable or authorized interested entities and combinations thereof are potential users of the present invention. Compliance monitoring may further include receiving prescription data associated with a dispensed prescription, the DEA Registration number associated with the prescriber, and determining whether the patient is compliant by analyzing prescription data. Compliance monitoring may, also include issuing an indication or notification of whether the patient is compliant to the prescriber.

### BRIEF DESCRIPTION OF THE DRAWINGS

FIG. 1 is an explanatory block diagram of the monitoring system for drug distribution.

FIG. 2 shows a flow chart of an example of a method of tracking drugs in accordance with one embodiment of the invention.

FIG. 3 is an explanatory block diagram of one embodiment of the tracking unit used in the monitoring system for drug distribution.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

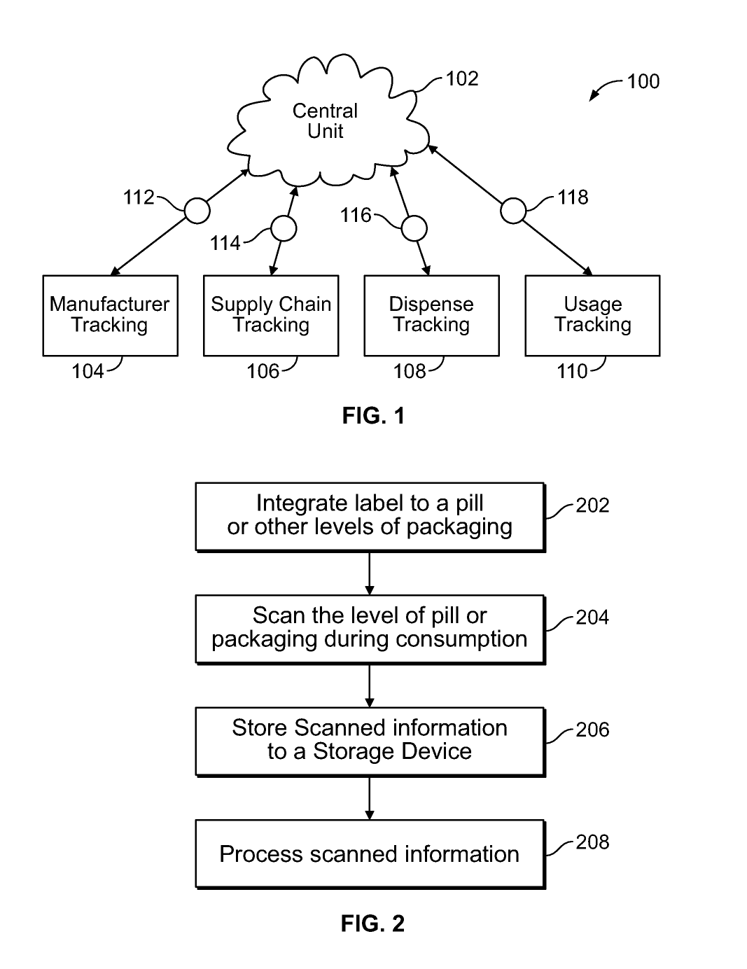

FIG. 1 shows an explanatory block diagram of the monitoring system for drug distribution. In FIG. 1, one embodiment of the systems with the components of the system for drug tracking and/or patient compliance monitoring is described herein. Controlled substances and other drugs are a medical necessity with potential for abuse and misuse, expiration and verifiable disposal and subject to recall. As a result, regulating controlled drugs is a substantial component of federal U.S. drug policy, and encompasses regulation of manufacture, importation, possession, use and distribution of these drugs.

In FIG. 1, the central unit 102 communicates with other illustrated tracking units 104-110. The central unit 102 receives and stores medical and/or non-medical information regarding the drugs and personnel throughout the tracking process of this illustrated embodiment of the present invention. Such information could include, but not limited to drug manufacturer, manufacturing dates and location, prescription data, dispense data, prescriber, pharmacy, patient, and law enforcement information. The central unit 102 can process these received and stored information in order to determine potential abuse and misuse, expiration and verifiable disposal and recall information etc.

Accordingly, the present invention described herein improve available tracking, compliance and authentication techniques by providing a unified or cohesive monitoring system instead of the disparate systems currently in use. In one embodiment, the central unit 102 has a central database that is used to track a drug through a product's lifecycle from manufacture to use by a patient. At any given point in the lifecycle of a drug, the system of the present invention is adapted to recognize inconsistencies or patterns indicating illicit or improper use.

Large numbers of controlled drugs are routinely made available for dispensing via prescriptions and generally include opioids such as codeine, hydrocodone, oxycodone or psychostimulants such as methylphenidate and amphetamine, opioid intermediates, depressants such as some barbiturates, benzodiazepines, narcotics, steroids such as anabolic steroids, and/or the like.

In one embodiment, a centralized system 100 is configured to monitor a drug as it travels from manufacturer through the manufacturer tracking unit 104, to distributor through the supply chain tracking unit 106, to pharmacy through the dispense tracking unit 108, and the prescriber (doctor) as well as the patient through the usage tracking unit 110. At each step in the lifecycle, the system 100 is programmed with the ability to spot inconsistencies or patterns of improper use.

For example, the system 100 is configured to monitor and compare the quantities of particular drug flowing in the system 100 from a manufacturer to the patient utilizing the various illustrated tracking units 104-110. At any point in the distribution chain, when there is a spike in the quantity of a product either being shipped, stored or dispensed, that does not match the known quantity of authorized manufacture, a signal may be generated by the appropriate tracking unit indicating the entrance of a counterfeit product. Such signal may be communicated to the central unit 102. After processed by the central unit 102, an alert message may be communicated to a prescriber, pharmacy, manufacturer, and/or law enforcement.

One feature of the present invention is that it monitors the global quantities of a drug moving through the supply chain to conduct an intelligent analysis. This requires an efficient method to quickly and efficiently track a drug from large quantity shipments to individual or unit dose dispensing. The teachings of U.S. Pat. No. 6,861,954 may be implemented to accomplish this needed feature. The disclosure of this patent is incorporated herein by reference in its entirety.

Once a controlled drug enters the supply chain there is a need to maintain the ability to effectively track the drug from the container level to the unit dosage level. In the illustrated embodiment, the supply chain tracking unit 106, dispense tracking unit 108, and usage tracking units 110 may all include the tracking on the container level to the unit dosage level. Detailed tracking unit components and the method used to track such information will be illustrated hereinafter. The illustrated system 100 can also monitor patient compliance, counterfeiting, and diversion, using the various tracking units 104-110 and the central unit 102.

The central unit 102 can also store and process authorized sales information. When the amount of authorized sales is less than the known quantity of authorized manufacture of a product or that stored in a particular location, a signal may be generated by the central unit 102 indicating unauthorized diversion of a product. By monitoring the overall state of the supply chain, points of potential problems may be identified and investigated. Once potential counterfeiting or diversion is spotted, a signal may be generated by the central unit 102 indicating the entrance of a counterfeit product or an improper diversion. Such signal may be provided to the relevant authorities for further investigation or processing.

Another problem the present invention addresses is doctor shopping. Because the system 100 receives patient prescription data, it is able to monitor and track the number of prescribers, by using for example DEA numbers, from which a particular patient obtains a particular subscription for a particular ailment. Once an irregularity is detected through the usage tracking unit 110, a signal may be generated notifying past and future prescribers as well as other appropriate personnel.

Similarly, based again on patient prescription data, the system 100 is also able to monitor and track the number of prescriptions a particular patient obtains for a certain drug. If the volume obtained is a mismatch with known treatment regimes, the system is designed to detect this irregularity through the usage tracking unit 110. Again, a signal may be generated by the central unit 102 notifying past and future prescribers as well as other appropriate personnel that the individual may be obtaining excessive quantities of a drug.

In another embodiment, the present invention provides a method wherein using the dispensing tracking unit 108, each time a prescription is filled the prescribing physician or entity may be notified electronically by computer or other media. The dispensing entity could then verify that the patient is his, or this could be done automatically via comparison with a database. Should the prescription be unauthorized, for example, if the patient was not a patient of that prescribing entity or if the prescriber never wrote the prescription, or if the number of pills was forged, then the prescriber would be notified and depending upon preference, DEA or law enforcement will be notified as well. Local pharmacies or chain pharmacies may also be notified and computerized algorithms may be used to prevent further forgeries.

In addition, by notifying the prescriber, the prescriber is able to compare the number of authorized prescriptions issued with the number detected. A mismatch may indicate potential forgeries. Once such determination is made, the information may be received and stored by the system 100 for future use.

In addition, prescriber information may be obtained by the system 100 and may be monitored, both at the issuing level and receipt by a dispenser. Such monitoring may be performed by the dispense tracking unit 108. Again, an inconstancy may detect forged prescriptions.

Moreover, the system 100 by monitoring the number of prescriptions issued to an individual for a particular drug via the usage tracking unit 110, other patterns of misuse may be detected. These may include prescribers stepping outside the bounds of known treatment protocols, detected via the dispense tracking unit 108.

In addition, the system 100 is also designed to detect individual non-compliance instances such as forged prescriptions, counterfeit products and diversion as discussed in further detail below.

In yet other embodiments, the present invention provides privacy protection, that as applications of RFID become more accepted, provides one or more levels of verification to allow scanning only by one or more authorized parties and selectively provides various levels information associated with a particular code. In one embodiment, a simple scan like an EZ pass, could violate patient privacy. To address privacy concerns, the system 100 may include authentication/authorization units 112-118. The scanner in each tracking unit 104-110 may be configured to transmit a code to the RFID device in a pill, bottle, case or other holder. The RFID device containing an actuating chip or other verification technology would only transmit certain information appropriate for the received code. Therefore, the current invention embodies compliance with HIPPA, and other privacy requirements regarding patient medical data, or other personal information, and provides signal or multilevel access points or modalities to improve pharma, device personal and other logistics, including decreasing drug diversion, controlled substances related morbidity and mortality and optimizing medical care.

Hence one or more codes, or level of code can be present in a unit of a medical product be it a pill, patch, bottle or the like. For example, in case of an overdose of an unknown medication in a patient presenting to an ER, an ER scanning device could transmit and unlock the RFID to transmit medication and strength and number of pills in the overdosed patient's body. If Drug diversion is suspected, a warrant could may be obtained and a higher level of verification code transmitted by the scanner to the RFID device to unlock the name of the person to whom the pill was prescribed which would then be transmitted to the scanner. If the patient is A and the medication was prescribed to person B, then diversion would be determined. Further, the dispensing pharmacy and all logistics would be available.

Similar processes could be utilized to track, maintain optimum function of, and allow required, scheduled or unanticipated medical devices such as pacemakers, spinal infusion pumps, stimulators, insulin pumps or to optimize animal or human surveillance of health status, function, location and the like.

In yet other embodiments, scanners may be located on roadways and toll booths, places of commerce, recreation and the like would be able to access information on RFID devices associated with medications, biologics and medical devices to monitor quantity and distribution.

FIG. 2 shows a flow chart of an example of a method of tracking medical products in accordance with one embodiment of the invention. Processing block 202 provides that labels are integral to a pill and one or more other levels of packaging. Processing block 204 provides that the usage tracking unit 110 illustrated in FIG. 1 using a scanning device to scan the label of a pill or the packaging during the consumption of the drug. Other indicia or indicating devices that may be used include the pill itself or some other part of ingested medication. The scanning device may be a patch or other device that is to be worn or carried by the person, including the indicia or indicator in the pill or in close proximity to a patient, permits for patient scanning. The scanned information is stored to a storage device as shown in block 206. Such stored information is readable by a reader, and may be transmitted to the central unit 102 for further processing as illustrated in FIG. 1. This step is illustrated in block 208.

For example, should a person present to an ER or be pulled over for a violation, physicians, EMTs or police or DEA officials could scan the patient to determine what medications the patient had consumed. Further, if the identifying data contained in the ingested medication pills, or applied patches or other unit doses, were prescribed to another individual this would indicate diversion. As illustrated in block 208, by using the age RFID pedigree the source of diversion would be readily discoverable.

In another embodiment, block 204 can be implemented by attaching the bottle to or be integral with a transmitter which can transmit data each time a pill or unit dose is removed. This can be done by weight or by each pill or dose having an RFID label integral to it.

The use of the pills could be tracked by this manner illustrated in FIG. 2 which could be voluntary or involuntary by variation. Processing of the information as illustrated in block 208 can be done locally at the usage tracking unit 110 as illustrated in FIG. 1. Or the stored information may be transmitted by a transmitter to the central unit 102 as illustrated in FIG. 1 for further processing. The transmitter could be in the form of a credit card or other small device which would need to be presented to a pharmacy in order for the pharmacist to dispense medications, or to the prescriber and this may be linked to either the bottle or pills or both.

The data contained in the storage device could be downloaded or otherwise transmitted at the time of the patient visit or telephonically or via Internet or other means in real time to the pharmacist, prescriber or others to keep track of the patient's use of the medication, and to the manufacturer who could keep the patient data in their database for the prevention of drug abuse.

The processing illustrated in block 208 may include drug abuse detection. For example, if the patient were to remove too many pills in too short a time, the physician, pharmacy or other individuals could be notified immediately through the alert sent out by the central unit 102 illustrated in FIG. 1. A mechanism could be incorporated such that any break in transmission or scheduled transmissions from the transmitter would also generate a signal notifying potential tampering.

Next a pedigree would be generated for the pills via the tracking units 104-110 illustrated in FIG. 1. Pills that were illegally obtained and these could be traced by a passive or active scanning and identified much like a lost telephone using the RFID technology described above or by using Internet technology or by scanning through totals. The pedigree may be generated using the process described in block 208 illustrated in FIG. 2.

An important part of this invention involves computerized algorithms and Internet, telephonic or other electronic data collection and processing with comparisons to note irregularities and this could extend down to the end-user i.e. the patient from the point of import or manufacture.

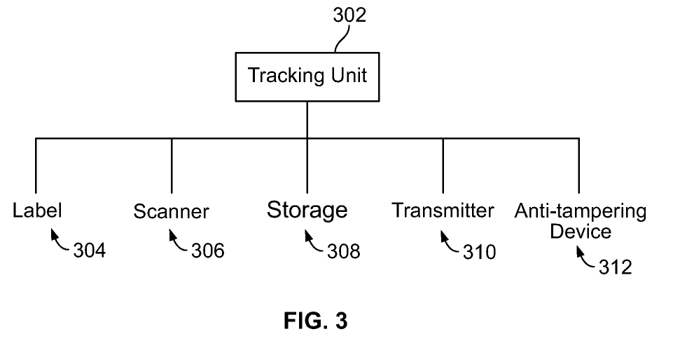

FIG. 3 is an explanatory block diagram of one embodiment of the tracking unit used in the monitoring system for drug distribution. The four tracking units 104-110 illustrated in FIG. 1 could have very similar components as illustrated in FIG. 3. The tracking unit 302 could be an example of any of the manufacturer tracking unit 104, or the supply chain tracking unit 106, or the dispense tracking unit 108, or the usage tracking unit 110.

Tracking unit 302 contains components such as at least one label unit 304 used to be integrated to a pill or other levels of packing of the drug. At least one scanning unit 306 used to scan the labels of the label unit 304. At least one storage unit to store the scanned information from the scanning unit 306. At least one transmitter to communicate with other tracking units and/or the central unit 102 illustrated in FIG. 1 of the system 100. The illustrated tracking unit 302 may also include at least one anti-tampering device 312, that detects and reports any tampering of the tracking unit 302.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiments, methods, and examples, but by all embodiments and methods within the scope and spirit of the disclosure.

What is claimed is:

1. A system for tracking, compliance and authentication of a mediation comprising:
a central unit adapted to track a drug through its lifecycle from manufacture to use by a patient;
a manufacturer tracking unit;
a supply chain tracking unit;
a dispense tracking unit; and
a usage tracking unit.

2. The system of claim 1 wherein said system adapted to recognize inconsistencies or patterns indicating illicit or improper use of a drug.

3. The system of claim 1 wherein said central unit comprises at least one storage, at least one processor, and at least one transmitter.

4. The system of claim 1 wherein said system further comprises at east one authentication unit.

5. The system of claim 1 wherein said usage tracking unit comprising:
at least one label;
at least one scanner;
at least one storage; and
at least one transmitter;

6. The system of claim 5 wherein said usage tracking unit further comprising at least one anti-tampering device.

7. A system of claim 1 wherein said provides one or more levels of verification to allow scanning only by one or more authorized parties and selectively provides various levels information associated with a code.

8. A method for tracking, compliance and authentication of a mediation comprising:
integrating at least one label to the medication;
scanning said at least one label;
storing said scanned label information to a storage device; and
processing said scanned information.

9. The method of claim 8 further comprising transmitting at least one alert signal to a receiver.

10. The method of claim 8 further comprising generating a pedigree of said medication.

11. The method of claim 8 further comprising authenticating at least one scanner via a predetermined code.

12. The method of claim 11 further comprising incorporating HIPPA requirements to said authenticating process.

13. The method of claim 10 further comprising detecting drug diversion using said generated pedigree of said medication.
